(19) United States
(12) Patent Application Publication
(10) Pub. No.: US 2012/0012108 A1
(43) Pub. Date: Jan. 19, 2012

(54) ARTIFICIAL NOSE AND BREATHING CIRCUIT PROVIDED WITH THE ARTIFICIAL AIRWAY

(76) Inventors: Yasuhiko Sata, Tokyo (JP); Norio Hachisu, Tokyo (JP)

(21) Appl. No.: 13/259,550

(22) PCT Filed: Mar. 11, 2010
(86) PCT No.: PCT/JP2010/054135

§ 371 Date: Sep. 23, 2011

(30) Foreign Application Priority Data
Apr. 6, 2009 (JP) 2009092150

(51) Int. Cl. A61M 16/16

(52) U.S. Cl. 128/203.14; 128/203.26

(57) ABSTRACT

Provided is an artificial airway and a breathing circuit provided with the artificial airway, including a tubular outer shell; a moisture permeable and water resistant film disposed on an entire circumference of an internal surface of the outer shell, forming a water retention region with the outer shell, and forming an aeration region on an internal surface side thereof; a feed water inlet provided in the outer shell to supply water to the water retention region; and a heater disposed outside the outer shell, heating the water in the water retention region to generate water vapor, and also heating an inspiratory gas flowing in the aeration region, wherein the water supplied from the feed water inlet is retained in the water retention region by the moisture permeable and water resistant film, and only the water vapor generated by the heating of the heater passes through the moisture permeable and water resistant film and flows into the aeration region to heat and humidify the inspiratory gas flowing in the aeration region.

### TECHNICAL FIELD

The present invention relates to an artificial airway and a breathing circuit provided with the artificial airway, and in particular, relates to an artificial airway and a breathing circuit to supply a heated and humidified inspiratory gas to a user.

### BACKGROUND ART

In a case of carrying out artificial respiration using a breathing circuit provided with an artificial airway, an inspiratory gas supplied to a person is required to be heated and humidified in advance. To deal with this, as shown in FIG. 5, a container 134 for heating and humidification having water stored therein is normally heated with a heater device 136 to generate water vapor and to pass an inspiratory gas to be supplied to a person through the container 134, thereby heating and humidifying it. However, after passing through the container 134, the inspiratory gas is cooled to recondense the water vapor while passing through a breathing circuit (inspiratory tube) 102, so that there arises a problem of not being able to supply an inspiratory gas sufficiently heated and humidified to a person. On the contrary, in order to supply an inspiratory gas at the optimal temperature and humidity to a person, the inspiratory gas is required to be heated up to a considerably high temperature at the time of passing through the container 134 for heating and humidification on the advance assumption of a temperature drop (refer to a graph in FIG. 5). It is also required to provide a water trap that collects the water recondensed in the breathing circuit (inspiratory tube) 102 and to provide a dew condensation preventing heater wire 140 in the breathing circuit (inspiratory tube) 102 to prevent water vapor from being recondensed.

Further, it requires excessive devices and members, such as the container 134 for heating and humidification and the heater device 136, and also requires a disposable humidifier connecting tube 138 to link an inspiratory gas supply source (respirator) 122 and the container 134 for heating and humidification, so that there arises a problem of rising facility costs and running costs. In addition, since connecting tubes are increased, there also arises a problem of increasing risks of a tube connection failure and disengagement of a tube.

To address these problems, humidification devices for a breathing circuit are proposed that are provided with a hollow fiber or a pipe having moisture permeability and water resistance, which is permeable to water vapor but not permeable to water, which is a liquid, disposed inside the breathing circuit (for example, refer to Patent Documents 1 through 3).

### PRIOR ART DOCUMENTS

### Patent Documents

Patent Document 1: Japanese Unexamined Patent Publication No. 2006-223332
Patent Document 2: Japanese Unexamined Patent Publication No. H9-122242
Patent Document 3: Japanese Unexamined Patent Publication No. S62-26076

### SUMMARY OF THE INVENTION

### Problems to be Solved by the Invention

In the device described in Patent Document 1 or 2, as shown in FIG. 6, water is supplied into a hollow fiber 150 having moisture permeability and water resistance to make water vapor generated by heating using a heater 152 arranged in the proximity of the hollow fiber 150 permeate to outside the hollow fiber 150, thereby humidifying the inspiratory gas flowing in the breathing circuit (inspiratory tube) 102, and at the same time, heating the inspiratory gas. Similarly, in the device described in Patent Document 3, water is supplied into a pipe having moisture permeability and water resistance to make vapor generated by heating using a heater disposed in the pipe permeate to outside the pipe, thereby humidifying the inspiratory gas flowing in the breathing circuit (inspiratory tube), and at the same time, heating it.

Therefore, the inspiratory gas can be humidified at a position nearer to a user compared with a case of using a container for humidification, so that they have an advantage regarding the problem of recondensation of water vapor in the breathing circuit (inspiratory tube). In addition, excessive devices, such as a container for humidification and a heater device, and a disposable connection tube becomes unnecessary, so that the facility costs and the running costs can be prevented from rising and the risks of a tube connection failure and disengagement of a tube can be reduced.

However, since a heating and humidifying mechanism (a hollow fiber, a pipe, a heater, and the like) is disposed inside the breathing circuit, the circuit resistance of the breathing circuit is increased and there is a possibility that the ventilation control and the airway internal pressure measurement will go wrong. In addition, a load on the inspiratory gas supply source is increased and there is a possibility that the running costs of the breathing circuit are increased. In particular, it is required to secure a heating and humidifying area by elongating the total length of the heating and humidifying mechanism to be sufficiently heated and humidified, so that the circuit resistance of the breathing circuit tends to be increased.

In addition, the heating and humidifying mechanism inside the breathing circuit makes contact with a wall of the breathing circuit and the inspiratory gas flows over there, and thus there is also a possibility of causing variation in heating and humidification. Further, as shown in FIG. 6, there is also a possibility of developing condensation of water vapor on the internal wall of the inspiratory tube 102 to cause a problem of retaining dew condensed water in the circuit.

Accordingly, it is an object of the present invention to provide an artificial airway that solves the problems mentioned above and has a simple configuration allowing to achieve heating and humidifying the inspiratory gas sufficiently for a user without increasing the flow resistance (circuit resistance) of the inspiratory gas within the artificial airway, and further, being less affected by a change in temperature from the outside, and without developing condensation on the wall of the circuit, and to provide a breathing circuit provided with the artificial airway.

### Means for Solving the Problems

To solve the problems mentioned above, one embodiment of an artificial airway of the present invention used for the breathing circuit is an artificial airway used for a breathing circuit, includes: a tubular outer shell; a moisture permeable and water resistant film disposed on an entire circumference of an internal surface of the outer shell, forming a water retention region with the outer shell, and forming an aeration region on an internal surface side thereof; a feed water inlet provided in the outer shell to supply water to the water retention region; and a heater disposed outside the outer shell, heating the water in the water retention region to generate water vapor, and also heating an inspiratory gas flowing in the aeration region, wherein the water supplied from the feed water inlet is retained in the water retention region by the moisture permeable and water resistant film, and only the water vapor generated by the heating of the heater passes through the moisture permeable and water resistant film and flows into the aeration region to heat and humidify the inspiratory gas flowing in the aeration region.

According to this embodiment, an inspiratory gas can be heated and humidified in the artificial airway arranged at a position nearer to a user, so that it is less affected by a change in temperature from the outside and the risks of recondensing water vapor within the artificial airway can be reduced. In addition, it does not require excessive devices and members, such as a container for heating and humidification, a heater device to warm water in the heating and humidifying container, and a device for controlling the amount of water and the temperature, and an excessive disposable connection tube is also not required, so that the facility costs and the running costs can be reduced and the risks of a tube connection failure and disengagement of a tube can also be reduced.

Further, the inspiratory gas can be heated and humidified using a large heating and humidifying area, such as the entire circumference of the internal surface of the outer shell of the artificial airway, so that heating and humidification of the inspiratory gas sufficient for a user can be realized and the condensation on the wall of the circuit is also not developed. In addition, since there is no excessive member for humidification in the artificial airway, there is also no possibility of increasing the flow resistance of the inspiratory gas and no possibility of having a ventilation control and measurement of an airway pressure gone wrong.

Another embodiment of the artificial airway of the present invention used for the breathing circuit is, further, the artificial airway, wherein the heater is disposed outside the outer shell in a region where the water retention region is formed.

According to this embodiment, a heater is disposed in a region where a water retention region is formed, so that the water stored in the water retention region can be heated sufficiently to generate water vapor, and further, the inspiratory gas can be humidified using a sufficient humidifying area corresponding to the water retention region. Similarly, the inspiratory gas passing through an aeration region can be heated using a sufficient heating area corresponding to the humidifying area.

Another embodiment of the artificial airway of the present invention used for the breathing circuit is, further, the artificial airway, wherein the heating and humidification of the inspiratory gas is possible to be adjusted at the same time by adjusting a power application to the heater.

Suppose if the flow rate of the inspiratory gas flowing in the aeration region increases, the amount of water vapor and the amount of heat to be added to the inspiratory gas are required to be increased, and if, on the contrary, the flow rate of the inspiratory gas decreases, the amount of water vapor and the amount of heat to be added to the inspiratory gas are required to be reduced. That is, the amount of water vapor and the amount of heat to be added to the inspiratory gas have positive correlation. Accordingly, as this embodiment, by adjusting the application power of one heater, the heating and humidification of the inspiratory gas can be adjusted at the same time, and thus the device configuration and the control process can be simplified.

Another embodiment of the artificial airway of the present invention used for the breathing circuit is, further, the artificial airway, wherein the moisture permeable and water resistant film is made of a resinous sheet or a resinous film.

According to this embodiment, by using a resin material, a highly reliable moisture permeable and water resistant film can be obtained.

Another embodiment of the artificial airway of the present invention used for the breathing circuit is, further, the artificial airway, wherein the moisture permeable and water resistant film includes a nonwoven fabric or a film having moisture permeability and water resistance.

Here, “the moisture permeable and water resistant film includes a nonwoven fabric having moisture permeability and water resistance” includes a case of using a nonwoven fabric only and also includes a case of using a material having a nonwoven fabric and another member, such as a water absorbing polymer, for example, in combination. According to this embodiment, a film can be obtained that has sufficient moisture permeability and water resistance at relatively low production costs.

Another embodiment of the artificial airway of the present invention used for the breathing circuit is, further, the artificial airway, wherein the moisture permeable and water resistant film includes a porous material or a nonporous material.

Here, a porous material is a material having micropores that is not permeable to a water droplet but permeable to a gas, including water vapor. In contrast, a nonporous material does not have micropores permeable to a gas, a liquid, and a gas, and for example, moisture permeates the material from the surface in contact with a water droplet and diffuses therein and evaporates from the other surface, thereby exhibiting the moisture permeable and water resistant performance.

According to this embodiment, both a porous material and a nonporous material can be used as the moisture permeable and water resistant film, so that it is possible to select an optimal one as the moisture permeable and water resistant film from diverse materials.

Another embodiment of the artificial airway of the present invention used for the breathing circuit is, further, the artificial airway, wherein a tubular reinforcement member is disposed on the internal surface side of the moisture permeable and water resistant film to make contact with the internal surface.

According to this embodiment, even in a case a tube configured with a moisture permeable and water resistant film does not have the strength for maintaining a shape (for example, cylindrical shape) of securing the aeration region, a tubular reinforcement member is disposed so as to make contact with an internal surface of the moisture permeable and water resistant film, so that the tube configured with a moisture permeable and water resistant film can be maintained in the shape and the moisture permeable and water resistant film can be prevented from expanding inward to secure the aeration region in a sufficient size.

The cross-sectional shape of the aeration region secured by the tubular reinforcement member is not limited to a circular shape and can have any cross-sectional shape, including elliptical and polygonal shapes.

Another embodiment of the artificial airway of the present invention used for the breathing circuit is, further, the artificial airway, wherein a helical core is disposed in the water retention region between the outer shell and the moisture permeable and water resistant film and the water supplied from the feed water inlet flows along a helical flow channel formed with the helical core.

According to this embodiment, even in a case that a tube configured with a moisture permeable and water resistant film does not have the strength for maintaining a shape (for example, cylindrical shape) of securing an aeration region, a helical core is disposed in the water retention region, so that the tube configured with a moisture permeable and water resistant film can be maintained in the shape and the moisture permeable and water resistant film can be prevented from expanding inward to secure the aeration region in a sufficient size. Since water flows along a helical flow channel formed with the helical core, the helical core does not impede the flow of the water in the water retention region.

The cross-sectional shape of the aeration region secured by the helical core is not limited to a circular shape and can have any cross-sectional shape, including elliptical and polygonal shapes.

Another embodiment of the artificial airway of the present invention used for the breathing circuit includes: an outer shell in an approximately cylindrical shape; a moisture permeable and water resistant film, formed into folds, disposed on an entire circumference of an internal surface of the outer shell, forming a water retention region with the outer shell, and forming an aeration region on an internal surface side thereof; a feed water inlet provided in the outer shell to supply water to the water retention region; and a heater provided in the water retention region or outside the outer shell, heating the water in the water retention region to generate water vapor, and also heating an inspiratory gas flowing in the aeration region, said artificial airway applicable as an artificial nose in which the inspiratory gas and an expiratory gas flow in the aeration region, wherein the water supplied from the feed water inlet is retained in the water retention region by the moisture permeable and water resistant film, and only the water vapor generated by the heating of the heater passes through the moisture permeable and water resistant film and flows into the aeration region to heat and humidify the inspiratory gas flowing in the aeration region.

According to this embodiment, the moisture permeable and water resistant film is formed into folds as a nasal cavity of a person, so that the heating and humidifying area can be large, and even an artificial airway having a relatively short total length, such as an artificial nose, for example, can sufficiently heat and humidify the inspiratory gas.

One embodiment of a breathing circuit of the present invention is a breathing circuit, including: the artificial airway as described above; an inspiratory gas supply source supplying the inspiratory gas to the aeration region of the artificial airway connected thereto; and water supply means supplying the water to the water retention region with a basically constant static pressure via the feed water inlet, wherein the water retention region is supplemented with water by the water supply means in an amount of water corresponding to an amount of water vapor passed through the moisture permeable and water resistant film and flown out.

According to this embodiment, by applying a basically constant static pressure, the water retention region can be supplemented with water in an amount of water corresponding to the amount of water vapor that has gone out through the moisture permeable and water resistant film, so that a breathing circuit can be provided that is capable of humidifying an inspiratory gas stably for a long period of time without an excessive control or the like.

Another embodiment of a breathing circuit of the present invention is, further, the breathing circuit, wherein the water supply means supplies the water by dropping from a container that contains the water and includes: drop rate measurement means measuring a rate of the dropping; and control means carrying out a control process of issuing an alert, based on drop rate measurement data sent from the drop rate measurement means, when the drop rate exceeds a predetermined value or when the drop rate falls below a predetermined value.

According to this embodiment, a control process of issuing an alert is carried out when the drop rate from the container containing water exceeds a predetermined value, so that even if the moisture permeable and water resistant film is broken to cause an event of water leakage, it is possible to secure safety of the user by issuing an alert promptly. A control process of issuing an alert is also carried out when the drop rate from the container containing water falls below a predetermined value, so that even in a case that the water supply tank becomes empty or that water becomes not supplied to the artificial airway for some reason (for example, an obstruction of the tube), it is possible to secure safety of the user by issuing an alert promptly.

Another embodiment of a breathing circuit of the present invention is, further, the breathing circuit, further including temperature measurement means measuring a temperature of the inspiratory gas flowing in the aeration region in proximity of an exit of the inspiratory gas of the artificial airway, wherein the control means carrying out a control process of adjusting the power application of the heater based on temperature measurement data sent from the temperature measurement means.

According to this embodiment, the temperature is measured in the proximity of an exit of the inspiratory gas, which is near the user, and the power application of the heater is adjusted based on the temperature measurement data, so that it is possible to supply an inspiratory gas at an optimal temperature with a less temperature drop after heating by the heater.

### Effect of the Invention

As described above, an artificial airway and a breathing circuit of the present invention can achieve heating and humidification of an inspiratory gas sufficient for a user with a simple configuration without increasing the flow resistance (circuit resistance) of the inspiratory gas within the artificial airway, and further, being less affected by a change in temperature from the outside, and without developing condensation on the wall of the circuit.

### BRIEF DESCRIPTION OF THE DRAWINGS

FIGS. 1(a) and 1(b) are schematic views illustrating a structure of one embodiment of an artificial airway of the present invention used for a breathing circuit.

FIG. 2 is a schematic view illustrating a configuration of one embodiment of a breathing circuit provided with the artificial airway shown in FIGS. 1(a) and 1(b).

FIG. 3 is a schematic view illustrating fields of application of an artificial airway according to the present invention and a breathing circuit provided with the artificial airway.

FIGS. 4(a) and 4(b) are schematic views illustrating a structure of an embodiment that applies an artificial airway according to the present invention to an artificial nose.

FIG. 5 is a diagram schematically illustrating structures of a porous material and a nonporous material.

FIG. 6 is a schematic view illustrating a structure of an embodiment of an artificial airway using a nonporous material as a moisture permeable and water resistant film.

FIG. 7 is a schematic view illustrating a structure of an embodiment of an artificial airway having a tubular reinforcement member disposed so as to make contact with an internal surface of a moisture permeable and water resistant film.

FIG. 8 is a schematic view illustrating a structure of an embodiment of an artificial airway having a helical core disposed in a water retention region between an outer shell and a moisture permeable and water resistant film.

FIG. 9 is a schematic view illustrating a configuration of a breathing circuit provided with a conventional artificial airway.

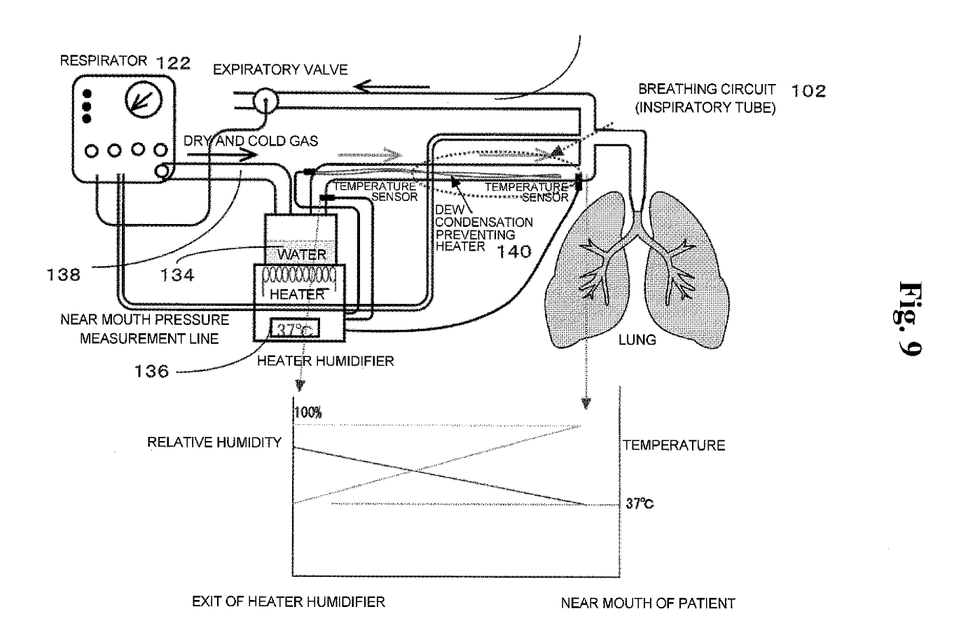

FIG. 10 is a schematic view illustrating a humidification device for a conventional breathing circuit in which a hollow fiber having moisture permeability and water resistance is disposed.

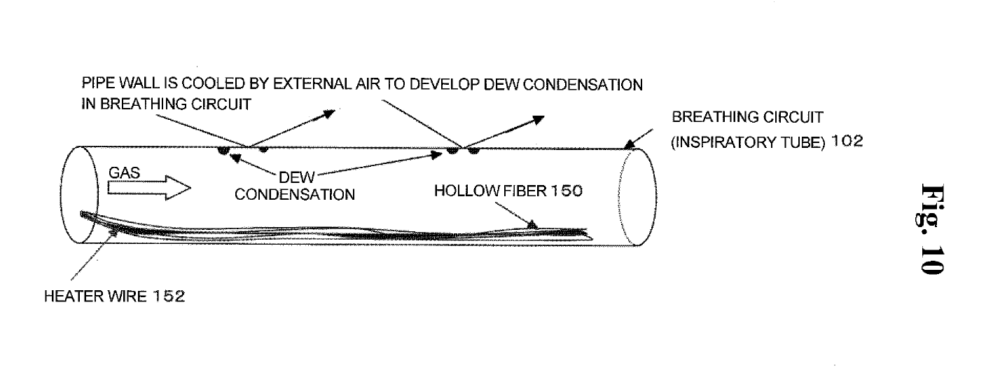

### MODE FOR CARRYING OUT THE INVENTION

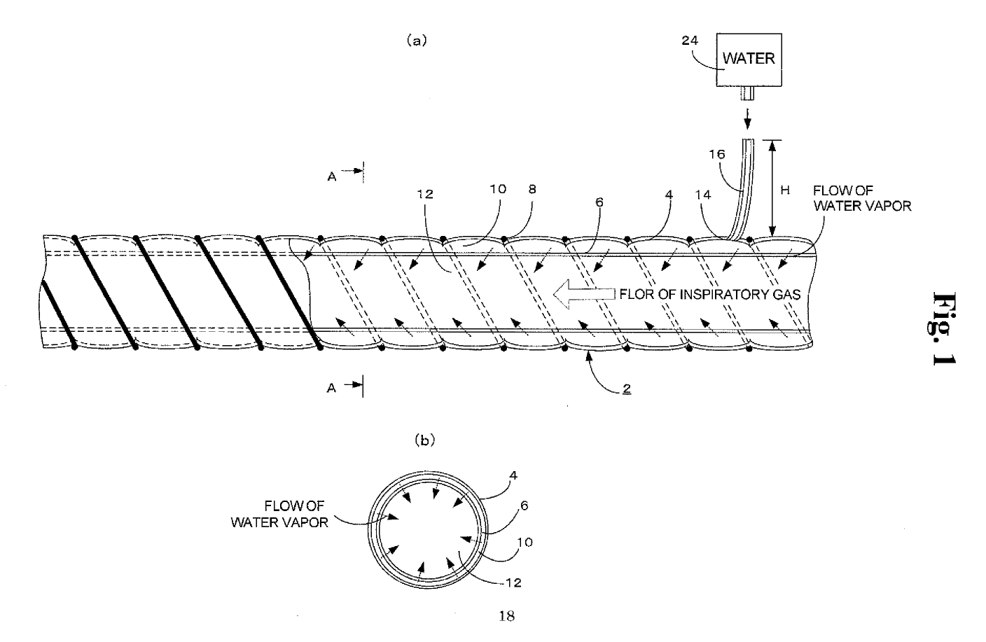

Embodiments of an artificial airway of the present invention used for a breathing circuit are described below with reference to the drawings. Here, FIGS. 1(a) and 1(b) are schematic views illustrating a structure of one embodiment of an artificial airway according to the present invention used for a breathing circuit, and

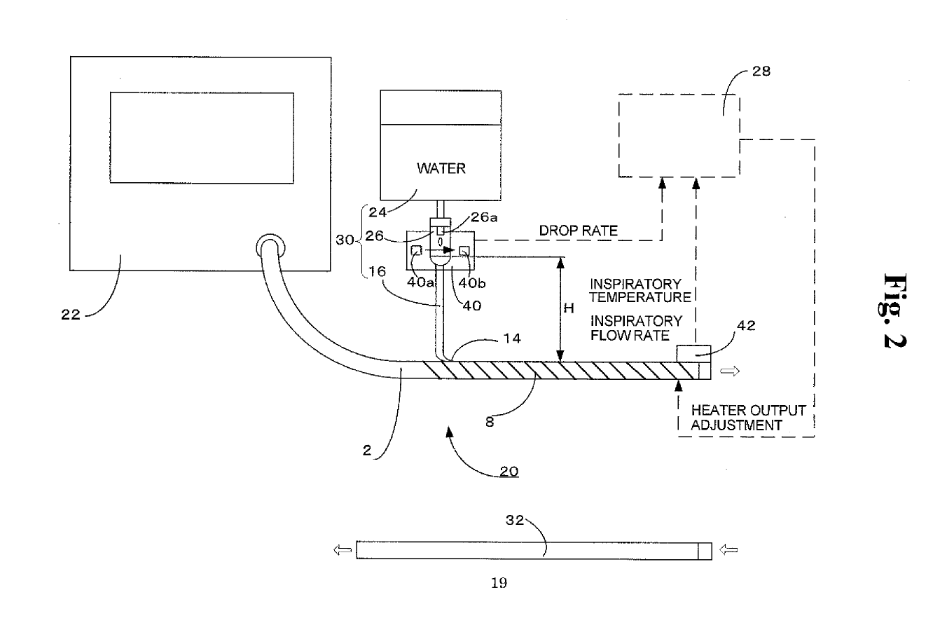

FIG. 2 is a schematic view illustrating a configuration of one embodiment of a breathing circuit provided with the artificial airway shown in FIGS. 1(a) and 1(b).

### (Description of One Embodiment of Artificial Airway According to the Invention)

Firstly, with reference to FIGS. 1(a) and 1(b), a detailed description is given to one embodiment of an artificial airway according to the present invention. Here, FIG. 1(a) is a schematic view of an artificial airway 2 taken from a side and illustrates a state of eliminating an outer shell 4 to expose the inside from the center to the right side in the drawing. FIG. 1(b) is a cross-sectional view taken from arrows A-A in FIG. 1(a).

An artificial airway 2 is provided with a tubular outer shell 4 having air tightness and water tightness, a moisture permeable and water resistant film 6 having moisture permeability and water resistance disposed on the entire circumference of the internal surface of the outer shell 4, and a heater 8 disposed outside the outer shell 4. Thus, a water retention region 10 is formed between the internal surface of the outer shell 4 and an outer surface of the moisture permeable and water resistant film 6, and an aeration region 12 is formed on the internal surface side of the moisture permeable and water resistant film 6. That is, the water retention region 10 and the aeration region 12 are partitioned by the moisture permeable and water resistant film 6.

As shown in FIG. 1(a), water supplied from a water container 24 is led into the water retention region 10 from a feed water inlet 14 through a water supply tube 16. In this case, at the feed water inlet 14, water is supplied to the water retention region 10 with a static pressure of a head of water H. The outer shell 4 has air tightness and water tightness, and the moisture permeable and water resistant film 6 has moisture permeability and water resistance which is permeable to a gas, like water vapor, but not permeable to water, which is a liquid, so that the water supplied from the feed water inlet 14 is retained in the water retention region 10 formed between the outer shell 4 and the moisture permeable and water resistant film 6.

The heater 8 of the present embodiment is a resistive heating linear heater (so-called ribbon heater) and is wound helically on an outer surface of the outer shell 4 in the entire region where the water retention region 10 is formed.

The artificial airway 2 with a configuration as described above has, as shown in FIG. 2, one end connected to an inspiratory gas supply source 22 configuring a breathing circuit 20, and a predetermined flow rate of an inspiratory gas flows in the aeration region 12 of the artificial airway 2 to be supplied to a user. In FIG. 1(a), as shown with a hollow arrow, the inspiratory gas flows in the aeration region 12 from the right side to the left side of the drawing. Examples of the dimensions of this artificial airway 2 (that is, outer size of the outer shell 4) may have, for example, a length of from 800 to 2000 cm and an outer diameter of from 10 to 40 mm (for example, in the ISO standards, breathing circuit for children: 15 mm, breathing circuit for adults: 22 mm), but they are not limited thereto. Although the tubular outer shell 4 is normally in a cylindrical shape having a circular cross-sectional shape, it is not limited thereto and such a tubular shape also includes a case of having, for example, an elliptical or polygonal cross-sectional shape.

A predetermined power is supplied to the heater 8 in a state where water is retained in the water retention region 10, thereby heating the water retained in the water retention region 10 to generate water vapor. The generated water vapor permeates the moisture permeable and water resistant film 6 as shown with arrows in broken lines in FIGS. 1(a) and 1(b) and flows into the aeration region 12 to be incorporated in the inspiratory gas flowing in the aeration region 12. Thus, the inspiratory gas can be heated and humidified.

At the same time to this, the heater 8 can give not only the water in the water retention region 10 but also a predetermined amount of heat to the inspiratory gas flowing in the aeration region 12, so that the inspiratory gas can also be heated. That is, in the present embodiment, it is possible to heat and humidify the inspiratory gas at the same time by the heater 8.

Suppose if the flow rate of the inspiratory gas flowing in the aeration region 12 increases, the amount of heat and the amount of water vapor to be added to the inspiratory gas is required to be increased, and if the flow rate of the inspiratory gas decreases, the amount of heat and the amount of water vapor to be added to the inspiratory gas is required to be reduced. That is, the amount of heat and the amount of water vapor to be added to the inspiratory gas have positive correlation. Accordingly, as the present embodiment, the heating and humidification of the inspiratory gas can be adjusted at the same time by adjusting the power application of the one heater 8, and thus the device configuration and the control process can be simplified.

In the present embodiment, the heater 8 is disposed outside the outer shell 4 in the entire region where the water retention region 10 is formed. This enables the water stored in the water retention region 10 to be heated sufficiently to generate water vapor, and further, to humidify the inspiratory gas using the sufficient humidifying area corresponding to the water retention region 10. Similarly, by using the sufficient heating area corresponding to the humidifying area, the inspiratory gas passing through the aeration region 12 can be heated.

A detailed description is give below to components configuring the artificial airway 2.

### <Description of Outer Shell 4>

The outer shell 4 is configured with a resin material having air tightness and water tightness and also flexibility, and in the present embodiment, it is configured with vinyl chloride. It should be noted that it is not limited thereto and any other resin material, including polypropylene, polyethylene, polyethylene and ethylene vinyl acetate, and polyvinyl chloride, can be used.

The outer shell 4 of the present embodiment is formed with a helical recess, and along this helical recess, the linear heater 8 wraps around the outer surface of the outer shell 4. Employing such a configuration enables the heater 8 to be disposed evenly on the entire circumference of the outer shell 4 of the water retention region 10. This enables to realize even heating of the water and the inspiratory gas in the entire area of the water retention region 10. It should be noted that the shape of the outer surface of the outer shell 4 is not limited thereto and it can also have a flat outer surface with no recess and protrusion.

### <Description of Moisture Permeable and Water Resistant Film 6>

The moisture permeable and water resistant film 6 of the present embodiment is configured with a moisture permeable and water resistant sheet or a moisture permeable and water resistant film, and can be formed by rolling this sheet/film in a tubular shape to a diameter slightly smaller than the inner diameter of the outer shell 4 and seal bonding the both ends in the total longitudinal length. This moisture permeable and water resistant film 6 in a tubular shape is inserted into the outer shell 4 and the outer shell 4 and the moisture permeable and water resistant film 6 are seal bonded at the both longitudinal ends of the outer shell 4, thereby enabling to form the structure shown in FIGS. 1(a) and 1(b). These seal bondings can be realized using an adhesive.

The static pressure (for example, head of water H=100 cm H₂O) applied to the water retention region 10 is not high, so that the moisture permeable and water resistant film 6 is considered to obtain sufficient rigidity by bonding at the both longitudinal ends of the tubular outer shell 4 while it is also possible to spot bond the outer shell 4 and the moisture permeable and water resistant film 6 with a predetermined pitch as needed.

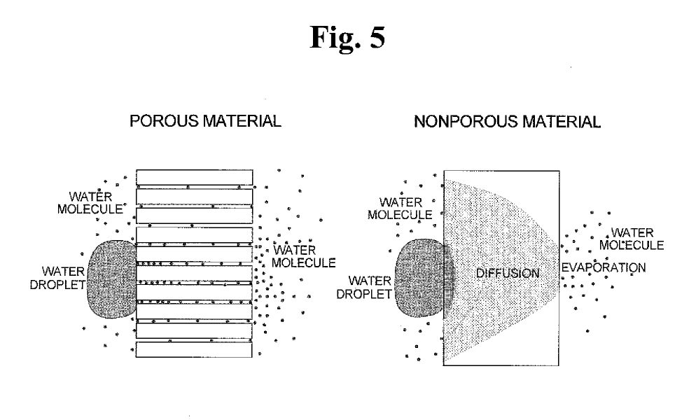

The moisture permeable and water resistant sheet/film used for the moisture permeable and water resistant film 6 is required to have a moisture permeable performance that is sufficiently permeable to water vapor and a water pressure resistant performance that can sufficiently withstand the applied water pressure. As a moisture permeable and water resistant sheet/film requiring such performances, porous materials and nonporous materials as shown in FIG. 5 can be used.

As shown in a left drawing of FIG. 5, a porous material is a material having micropores that are not permeable to a water droplet but permeable to a gas, and the micropores are permeable to water vapor, which is a gas including water molecules. An amount of permeating water vapor is determined by a humidity difference and a temperature difference between the spaces on both sides interrupted by the porous material. That is, in the left drawing of FIG. 5, in a case that the humidity is low and the temperature is high in the right side region of the porous material, the amount of permeating water vapor increases.

Such a structure enables to have the moisture permeable performance that is sufficiently permeable to water vapor and the water pressure resistant performance that can sufficiently withstand the applied water pressure. Specific examples of a porous material may be the materials shown in Table 1 described later.

In contrast, as shown in a right drawing of FIG. 5, a nonporous material does not have the micropores that are permeable to liquids gases, and moisture permeates the material from the surface in contact with a water droplet and diffuses therein and evaporates from the other surface, thereby exhibiting a moisture permeable and water resistant performance. The amount of permeating water vapor is determined by a temperature difference between the spaces on the both sides interrupted by the porous material. That is, in the right drawing of FIG. 5, in a case that the temperature in the right side region of the porous material is high, the amount of permeating water vapor increases.

Such a structure enables a nonporous material to have the moisture permeable performance that is sufficiently permeable to water vapor and the water pressure resistant performance that can sufficiently withstand the applied water pressure. Specific examples of a nonporous material may be a moisture permeable and water resistant sheet/film supplied by ARKEMA and a moisture permeable and water resistant sheet/film called SYMPATEX, a trade name, supplied by Akzo Nobel.

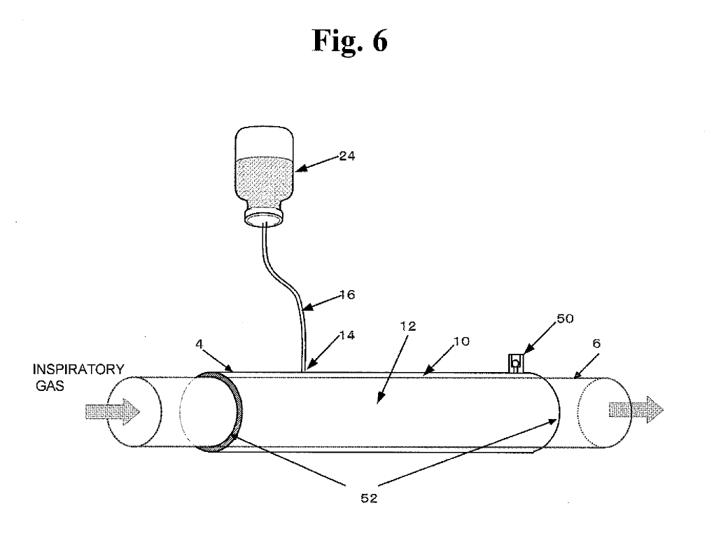

FIG. 6 illustrates an embodiment of the artificial airway 2 in a case of using a nonporous material as the moisture permeable and water resistant film 6. This artificial airway 2 is provided with the tubular outer shell 4 having air tightness and water tightness and the moisture permeable and water resistant film 6 including a nonporous material disposed on the entire circumference of the internal surface of the outer shell 4, and at both ends of the artificial airway 2, the outer shell 4 and the moisture permeable and water resistant film 6 are seal bonded by a sealing member 52. Thus, the water retention region 10 is formed between the internal surface of the outer shell 4 and the outer surface of the moisture permeable and water resistant film 6, and the aeration region 12 is formed on the internal surface side of the moisture permeable and water resistant film 6. Outside the outer shell 4, a heater is disposed (not shown).

The water stored in the water container 24 is led into the water retention region 10 from the feed water inlet 14 through the water supply tube 16. At this time, to make the water flow into the water retention region 10, it is required to exhaust the air present in the water retention region 10 to outside the water retention region 10 in advance. In this case, if the moisture permeable and water resistant film 6 were a porous material, the air could be exhausted through the micropores of the porous material, while if the moisture permeable and water resistant film 6 is a nonporous material, exhaustion cannot be carried out through the moisture permeable and water resistant film 6.

With that, the embodiment shown in FIG. 6 is provided with an exhaust outlet 50 to exhaust the air present in the water retention region 10 in advance via the exhaust outlet 50. This exhaust outlet 50 is provided with a check valve, which allows exhausting the air in the water retention region 10 but does not allow the external air to flow into the water retention region 10. Although FIG. 6 shows a ball check valve, it is not limited thereto and can use any other types of check valve.

In the present embodiment, by capping the exhaust outlet 50 after exhausting all air in the water retention region 10, the water in the water retention region 10 is kept from flowing out to outside. It should be noted that it is not limited thereto and the exhaust outlet 50 to flow the air but not to flow water can also be formed by, for example, putting a porous material on a top opening of the exhaust outlet 50.

It is also possible to put a highly hygroscopic material, such as a gel water absorbing and filter paper, for example, in the water retention region 10 formed between the outer shell 4 and the moisture permeable and water resistant film 6.

As described above, in the present embodiment, not only a porous material but also a nonporous material can be used as the moisture permeable and water resistant film 6 by being provided with the exhaust outlet 50, so that it is possible to select an optimal one as the moisture permeable and water resistant film 6 from diverse materials.

Next, the moisture permeable performance (degree of moisture permeability) and the water pressure resistant performance (water pressure resistance) required as the moisture permeable and water resistant film 6 are reviewed as below.

Ideal heating and humidifying conditions required for an artificial airway are generally to supply an inspiratory gas having a relative humidity of 100% (44 mg/L maximum) at a temperature of 37° C. to a user. Therefore, in the description below, a case, as an example, is calculated that an inspiratory gas at a temperature of 37° C. and at a relative humidity of 100% (44 mg/L maximum) is supplied at 6 L/min where an amount of breathing of an adult male is 6 L/min.

The maximum amount of water vapor to be supplied to the inspiratory gas by permeating the moisture permeable and water resistant film 6 for 24 hours becomes:

6 (L/min)×44 (mg/L)×60×24×1/1000=approximately 380 g/24

A humidifying area to make water vapor permeate (area of the moisture permeable and water resistant film 6) is considered to be, assuming that, for example, the water retention region 10 has an inner diameter of 2.2 cm and has a length of 100 cm, approximately 0.069 m² (=2.2/100×1×3.14).

Accordingly, 380 g/24 hrs of water vapor is required to permeate in the entire area of the moisture permeable and water resistant film 6 having a humidifying area of 0.069 m², so that a degree of moisture permeability of approximately 5,500 g/m2·24 hr (=380/0.069) is required for a moisture permeable and water resistant sheet/film used for the moisture permeable and water resistant film 6.

Then, the water pressure resistant performance (water pressure resistance) of the moisture permeable and water resistant film 6 is reviewed where the dimensions of H shown in FIG. 1(a) is considered to be approximately from 40 cm to 200 cm by considering specific arrangement of the artificial airway 2 and water supply means 30. Accordingly, 200 cm H₂O or more of water pressure resistance is considered to be required.

The moisture permeable performance required for actual use is, taking a safety factor of some extent into consideration, a degree of moisture permeability (JIS K 7129 (A method)) of preferably 6,000 g/m2.24 hr or more, more preferably 8,000 g/m2.24 hr or more, and even more preferably 10,000 g/m2.24 hr.

The water pressure resistance is, taking a safety factor of some extent into consideration, preferably 400 cm H₂O or more, more preferably 800 cm H₂O or more, and even more preferably 1000 cm H₂O or more.

Some examples of a specific material (porous material) having such a moisture permeable performance and a water pressure resistant performance are shown in the table below. In the table below, materials including resinous sheets/films and a nonwoven fabric are shown.

TABLE 1

| | | | Degree Of | Water | |
|---|---|---|---|---|---|
| | | | Moisture | Pressure | |
| | | | Permeability | Resist- | |
| | | Corporate | A Method | ance | |
| No. | Trade Name | Name | g/m2 · 24 hr | cm H₂O | Material |
| 1 | FGX Film | Hiramatsu | 14,000 | 3,000 | Polyurethane |
| | | Sangyo | | | Porous Film |
| | | Company | | | |
| 2 | GEOVIS | Toyocloth | 10,000 | 499 | Urethane |
| | OR-αD | Co., Ltd. | | | |
| 3 | AGX-3381 | Toyocloth | 10,240 | 1,200 | Polyurethane |
| | | Co., Ltd. | | | |
| 4 | Gore-Tex | Japan | 13,500 | 4,000 | Teflon |
| | XCR | Gore-Tex | | | |
| | | Inc. | | | |
| 5 | Microporous | Sumitomo | 12,000 | 1,000 | Polypropylene |
| | Film | 3M | | | based |
| | | Limited | | | Microporous |
| | | | | | Film |
| 6 | Mitsubishi | EXEPOL | 7,200 | 1,600 | Polyethylene |
| | Plastics, Inc. | | | | |

In a case of using a resinous material having the moisture permeable performance and the water pressure resistant performance (for example, the materials of from #1 to #5 in Table 1), it is possible to obtain a highly reliable moisture permeable and water resistant film 6. In a case of using a nonwoven fabric, it is possible to obtain a moisture permeable and water resistant film 6 at relatively low production costs. Since there is a possibility of large water leakage, once water permeates, from that spot in a case of a nonwoven fabric singly, it is preferred to use a material, for example, having a nonwoven fabric and a water absorbing polymer or the like in combination (for example, the material of #6 in Table 1).

It should be noted that the material including a moisture permeable and water resistant sheet/film and a nonwoven fabric used for the moisture permeable and water resistant film 6 is not limited to the materials including the resinous sheets/films and the nonwoven fabric mentioned above, and it is possible to use a material including any resinous sheet/film and nonwoven fabric having a predetermined moisture resistant performance and a predetermined water pressure resistant performance.

### <Description of Humidifying Area and Heating Area>

As described later, the humidifying area in a case of heating the conventional container 134 for humidification to humidify the inspiratory gas is considered (refer to FIG. 5) by assuming, for example, a circular heating surface having a diameter of 10 cm to obtain 0.008 m² (=10×10×3.14×1/4×1/10000). In contrast, the humidifying area in the present embodiment becomes approximately 0.069 m² assuming that the water retention region 10 has an inner diameter of 2.2 cm and has a length of 100 cm similar to above. Accordingly, in the present embodiment, a very large humidifying area can be obtained compared to a case of passing an inspiratory gas through a conventional heated container for humidification. At the same time, the inspiratory gas can be heated in the area same as this humidifying area, so that it is possible to obtain a very large heating area compared to a case of heating an inspiratory gas by passing through a container for humidification.

### <Description of Heater 8>

In the present embodiment a so-called ribbon heater (a nichrome wire coated by a fabric woven with heat resistant glass fibers) is used as the heater 8, so that it is excellent in flexibility and can easily wrap around along the recess helically formed on the outer surface of the outer shell 4. This enables to dispose the heater 8 evenly on the entire circumference of the outer shell 4 covering the water retention region 10 and it is possible to efficiently realize even heating of water and the inspiratory gas in the entire area of the water retention region 10. It should be noted that it is not limited thereto configuration and it is also possible to, for example, cover the outside of the outer shell 4 with a sheeted heater and to use any other heater.

Then, a specific heating capacity of the heater 8 is reviewed. As the above description, a case of generating water vapor at 380 g/24 hr is considered, assuming that the heat of vaporization of water at 20° C. (water temperature in the water retention region 10) is 586 cal/g and the thermal efficiency of the heater for the power application is 20%, to have the power application required for the heater being 380 (g/24 hrs)×586 (cal/g)×1/24×1/860 (cal/W·h)/0.2=54 W·hr.

Accordingly, taking a safety factor of some extent into consideration, it is considered that sufficient water vapor can be generated by applying power at approximately from 60 to 100 W·hr to the heater 8. In contrast, in a case of heating the inspiratory gas, the specific heat of the inspiratory gas is very low compared to the heat of vaporization of water, so that it is considered that the heating of an inspiratory gas can be covered sufficiently by applying power at approximately from 60 to 100 W·hr to the heater 8. The power applications are merely some examples, and the optimal heater capacity may be determined in accordance with the flow rate of the inspiratory gas and the range of the water retention region that are actually used. Where the flow rate of the inspiratory gas and the range of the water retention region are considered, it is considered to be preferred to provide the heater 8 with a capacity of approximately from 20 to 150 W.

### <Description of Balance of Heating and Humidification>

As the above description, since the amount of water vapor and the amount of heat to be added to the inspiratory gas have positive correlation, the heating and humidification of the inspiratory gas can be adjusted at the same time by adjusting the power application of one heater 8 as the present embodiment. However, since the amount of water vapor and the amount of heat to be added to the inspiratory gas cannot be adjusted individually, it is required to adjust the volume of the water retention region 10, the capacity of the heater 8, the humidifying area, the heating area, and the like in advance so as to balance the amount of water vapor and the amount of heat. That is, within the range of adjusting power applied to the heater 8, it is required to generate heating and humidification at a rate not causing a trouble for actual use.

For example, even with the same humidifying area and the same heating area, when the interval between the outer shell 4 and the moisture permeable and water resistant film 6 are different, the volume of the water retention region 10 changes, so that the amount of generated water vapor becomes different even if the same amount of power is applied to the heater 8. In a case of intending to increase the ratio of heating to humidification, it is also possible to dispose the heater 8 outside the outer shell 4 in a region where there is no water retention region 10. On the contrary, in a case of intending to increase the ratio of humidification to heating, it is also considered to use a highly thermally insulative material as the moisture permeable and water resistant film 6.

Adjusting various elements as above enables the heating and humidification of the inspiratory gas to be adjusted at the same time with no problem for actual use by adjusting the power application of one heater 8.

### <Description of Feed Water Inlet 14>

The feed water inlet 14 to supply water to the water retention region 10 can be formed by making a hole, in the outer shell 4, having a diameter approximately identical to an outer diameter of that of the water supply tube 16, by inserting the water supply tube 16 into this hole, and by seal bonding the outer circumference of the water supply tube 16 and the outer shell 4 using an adhesive. For the water supply tube 16, a resin material same as that of the outer shell 4 can be used and any other resin material can also be used.

As described above, according to the above embodiment, the inspiratory gas can be heated and humidified in the artificial airway 2 arranged at a position nearer to a user, so that it is less affected by a change in temperature from the outside and the risks of recondensing water vapor in the artificial airway 2 can be reduced. In addition, it does not require excessive devices and members, such as a container for heating and humidification, a heater device to warm water in the heating and humidifying container, and a device for controlling the amount of water and the temperature, and also not required for an excessive disposable connection tube, so that the facility costs and the running costs can be reduced and risks of a tube connection failure and disengagement of a tube can also be reduced.

Further, the inspiratory gas can be heated and humidified using a large heating and humidifying area, such as the entire circumference of the internal surface of the outer shell 4 of the artificial airway 2, so that the heating and humidification of the inspiratory gas sufficient for a user can be realized and it also does not develop condensation on the wall of the circuit. In addition, since there is no excessive member for humidification in the artificial airway 2, there is no possibility of increasing the flow resistance of the inspiratory gas and also no possibility of the ventilation control or the airway pressure measurement going wrong.

(Description of One Embodiment of Breathing Circuit Provided with Artificial Airway According to the Invention)

Then, with reference to FIG. 2, a detailed description is given to one embodiment of a breathing circuit provided with an artificial airway according to the present invention. Here, FIG. 2 is a diagram schematically illustrating each device configuring the breathing circuit 20, including the artificial airway 2.

The breathing circuit 20 of the present embodiment is provided mainly with the artificial airway 2, the inspiratory gas supply source 22 connected to the artificial airway 2, the water supply means 30 to supply water to the water retention region 10 of the artificial airway 2, measurement means 40 and 42, and control means 28.

Regarding the measurement means 40 and 42 and the control means 28 of the breathing circuit 20 of the present embodiment, the water supply means 30 is provided with drop rate detection means 40 that measures the drop rate and an end of the artificial airway 2 on the exit side of the an inspiratory gas is provided with temperature measurement means 42 that measures the temperature of the inspiratory gas. The control means 28 carries out a predetermined control process based on measurement data received from the measurement means.

By the breathing circuit 20 with a configuration as mentioned above, the inspiratory gas supplied from the inspiratory gas supply source 22 is supplied to a user through the artificial airway 2 and the expiratory gas of the user is discharged to the atmosphere through an expiratory tube 32.

A description is given below to each component device configuring the breathing circuit 20.

### <Description of Water Supply Means 30>

The water supply means 30 is provided with the water container 24 and a dropping chamber 26 having an upper portion in communication with the water container 24 and a lower portion in communication with the water supply tube 16. The upper portion of the dropping chamber 26 is provided with a pipe 26a in communication with the water container 24 and the water in the water container 24 is dropped from this pipe 26a and thus the water can be supplied to the water supply tube 16 connected to the water retention region 10 of the artificial airway 2. As already described using FIGS. 1(a) and 1(b), the water supplied to the water supply tube 16 is supplied to the water retention region 10 through the feed water inlet 14.

Firstly, a procedure of filling water in the water retention region 10 is described. As the water container 24 is attached, the water flows from the water container 24 into the water retention region 10 due to the water pressure. At this time, the air retained in the water retention region 10 permeates the moisture permeable and water resistant film 6 and escapes to the aeration region 12 side. As the inside of the water retention region 10 is filled with water, water does not flow out of the water container 24. After that, an amount of water corresponding to the amount of water vapor passed through the moisture permeable and water resistant film and come out to the aeration region 12 is dropped from the pipe 26a to be supplied to the water retention region 10.

On the contrary, although there is a possibility that the inspiratory gas permeates the moisture permeable and water resistant film 6 from the aeration region 12 side to enter into the water retention region 10, the maximum pressure in artificial respiration is 100 cm H₂O or less, so that a back flow of the gas does not occur as long as the water container 24 is positioned 100 cm or more above the breathing circuit (artificial airway 2) (in FIG. 2, H>=100 cm).

For the water supply tube 16 from the water container 24 to the artificial airway 2, it is preferred to use, for example, a thin tube like one used for transfusion. Increasing the flow resistance in the tube using a thin tube enables to prevent a back flow of a gas even more effectively.

To describe the dropping chamber 26 further in detail, due to the dropping of water from the pipe 26a, water is retained in the lower portion of the dropping chamber 26 to form a water surface at a predetermined level (level shown with H). Here, the level of the water surface formed in the dropping chamber 26 is arranged so as to be higher by the difference H in height relative to the artificial airway 2.

Suppose if the level of the water surface rises in the dropping chamber 26, the air pressure in the dropping chamber 26 rises and acts to decrease the hydrostatic pressure to be a factor for water droplet formation, so that the drop rate becomes late. In contrast, suppose if the level of the water surface falls in the dropping chamber 26, the air pressure in the dropping chamber 26 falls and acts to increase the hydrostatic pressure to be a factor for water droplet formation, so that the drop rate becomes fast. Accordingly, the dropping chamber 26 has a self-adjusting function that adjusts the drop rate so as to always make the level of the water surface constant.

As described above, the level fluctuation of the water surface in the dropping chamber 26 is extremely small compared to the difference H in height with the artificial airway 2 and there is also the flow resistance of the water supply tube 16, so that the water supply means 30 can supply water to the water retention region 10 of the artificial airway 2 at a basically constant static pressure (head of water H). This enables the water retention region 10 to be supplement with water by the water supply means 30 in the amount of water corresponding to the amount of water vapor that has become water vapor by being heated by the heater 8 in the water retention region 10 of the artificial airway 2 and passed through the moisture permeable and water resistant film to come out to the aeration region 12.

As described above, by applying an approximately constant static pressure (head of water H), the water retention region 10 can be supplemented with water in the amount of water corresponding to the amount of water vapor passing through the moisture permeable and water resistant film 6 and gone out, so that it becomes possible to provide the breathing circuit 20 capable of humidifying the inspiratory gas stably for a long period of time without an excessive control process.

### <Description of Drop Rate Measurement Means 40>

Then, a description is given to the drop rate measurement means 40 provided in the water supply means 30. The drop rate measurement means 40 is mounted on a side portion of the dropping chamber 26 and is arranged to drop a water droplet between a light emitting device 40a emitting a visible light at a predetermined wavelength and a light receiving device 40b. When a water droplet drops, a light incident to the light receiving device 40b from the light emitting device 40a (refer to an arrow in FIG. 2) is interrupted, so that the dropping of water can be sensed. Since a time interval between the drops can be measured by a timer built in the drop rate measurement means 40, it is possible to accurately measure the drop rate. Then, the data of the drop rate of water measured by the drop rate measurement means 40 is sent to the control means 28.

In the present embodiment, although the drop rate measurement means 40 using a visible light sensor is shown as an example, it is not limited thereto and drop rate measurement means using any other sensor, including an infrared sensor, is applicable.

### <Description of Inspiratory Temperature Measurement Means 42>

By the temperature measurement means 42 provided at an end of the artificial airway 2 on the exit side of the inspiratory gas, the temperature of the inspiratory gas flowing in the aeration region 12 of the artificial airway 2 can be measured. Then, the temperature measurement data is sent to the control means 28. Here, as the inspiratory temperature measurement means 42, any conventional sensor can be used.

### <Description of Control Means 28>

As the control means 28 of the present embodiment, a commercially available computer can also be used that is provided with a processor (CPU), memory devices (ROM and RAM), an external interface, a driving circuit, and the like.

### <<Control Over Drop Rate>>

The control means 28 carries out a control process of issuing a predetermined alert when the drop rate of water exceeds a predetermined value or when the drop rate falls below a predetermined value based on the drop rate measurement data sent from the drop rate measurement means 40. That is, as the amount of water flowing into the water retention region 10 of the artificial airway 2 increases for some reason, the level of the water surface of the dropping chamber 26 drops, and the drop rate rises due to the self-adjusting function included in the dropping chamber 26. On the contrary, as the amount of water flowing into the water retention region 10 of the artificial airway 2 decreases for some reason, the level of the water surface of the dropping chamber 26 rises, and the drop rate drops due to the self-adjusting function included in the dropping chamber 26. Also in a case that the water in the water container 24 becomes less, the drop rate in the dropping chamber 26 drops as well. In a case that this drop rate exceeds a predetermined value or a case that the drop rate falls below a predetermined value, a control process of issuing a predetermined alert is carried out by, for example, sounding an alarm, activating an indication lamp, or sending a signal to a hospital system.

Here, in a case that the drop rate exceeds a predetermined value, there is a high possibility that the moisture permeable and water resistant film 6 of the artificial airway 2 is damaged and the water in the water retention region 10 is leaked to the aeration region 12 side, so that promptly issuing an alert enables to prevent a user from drowning (choked by water entering into a trachea or a lung) before it happens to secure the safety of the user.

Also when the drop rate from the container containing water falls below a predetermined value, a control process of issuing an alert is carried out, so that even if the water supply tank becomes empty or water becomes not supplied to the water retention region 10 for an obstruction of the tube or the like, it is possible to issue an alert promptly to secure safety of the user.

### <<Control Over Inspiratory Gas Temperature>>

The control means 28 carries out a control process of adjusting the power application to the heater 8 so as to make the temperature of the inspiratory gas at a set value based on the temperature measurement data sent from the temperature measurement means 42 of the artificial airway 2. The temperature is measured in the proximity of the exit of the inspiratory gas, which is near a user, and the power application of the heater 8 is adjusted based on the temperature measurement data, so that the temperature drop after heating by the heater 8 is less and the inspiratory gas at an optimal temperature can be supplied to the user.

Based on the temperature measurement data sent from the temperature measurement means 42, in a case that the inspiratory gas exceeds a predetermined temperature (for example, 43° C.), a control process of issuing a high temperature alert can be carried out, and similarly in a case that the temperature of the inspiratory gas falls below a predetermined value due to cable disconnection of the heater or the like, a control process of issuing a low temperature alert can be carried out.

In the present embodiment, since there is a sufficient humidifying area, it is possible to realize heating and humidification of the inspiratory gas (for example, a gas temperature of 37° C. and a relative humidity of 100%) sufficient for a user by measuring only the gas temperature without measuring the flow rate of the inspiratory gas. It should be noted that a control of the flow rate of the inspiratory gas by further providing a flow rate sensor is also applicable to the present invention.

(Comparison with Conventional Breathing Circuit)

Then, an embodiment of the breathing circuit 20 according to the present invention shown in FIG. 2 is described in comparison with the conventional breathing circuit shown in FIG. 5.

In the conventional breathing circuit shown in FIG. 5, the container 134 for heating and humidification having water stored therein is heated with the heater device 136 to generate water vapor to pass an inspiratory gas through the container 134, thereby heating and humidifying the inspiratory gas.

In this case, after passing through the container 134, the inspiratory gas is cooled while passing through the breathing circuit 102 and the water vapor is recondensed, and there arises a problem of not being able to supply a sufficiently heated and humidified inspiratory gas to the user. It is also required to provide a water trap to collect water condensed in the breathing circuit 102 because the water vapor develops recondensation and to further provide a dew condensation preventing heater 140 in the breathing circuit to prevent the recondensation.

Further, it requires excessive devices and members, such as the container 134 for heating and humidification and the heater device 136, and also requires the disposable humidifier connecting tube 138 to link between the inspiratory gas supply source 122 and the container 134 for humidification and, as the above description, the dew condensation preventing heater 140 and the water trap, so that the facility costs and the running costs are prone to be higher. In addition, connecting tubes are increased, so that a problem of increasing risks of occurring a connection failure and tube disengagement arises.

In contrast, in the breathing circuit 20 according to the present invention shown in FIG. 2, the inspiratory gas can be humidified in the artificial airway 2 at a position nearer to a user than that of the conventional container 134 for heating and humidification, so that there is less possibility of recondensation of the water vapor, in the artificial airway 2, included in the inspiratory gas. In addition, excessive devices can be reduced, such as the container 134 for heating and humidification and the heater device 136, so that the facility costs for the entire breathing circuit can be reduced. In addition, the number of disposable connecting tubes can be reduced, so that the facility costs and the running costs can be reduced and the risks of a tube connection failure and disengagement of a tube can be reduced.

As already described, although heating and humidifying mechanisms are proposed (refer to Patent Documents 1 through 3) that humidifies an inspiratory gas flowing in a breathing circuit by supplying water into a hollow fiber or a pipe that is moisture permeable and water resistant to make the water vapor generated by heating using a heater permeate to outside the hollow fiber or the pipe, since the heating and humidifying mechanism is disposed inside the breathing circuit in these proposals, the circuit resistance of the breathing circuit increases and there is a possibility of the ventilation control and the airway pressure measurement going wrong. In addition, the load on the inspiratory gas supply source is increased and thus there is a possibility of increasing the running costs of the breathing circuit. In particular, it is required to secure the heating and humidifying area by elongating the total length of the heating and humidifying mechanism for sufficient heating and humidification, so that the circuit resistance of the breathing circuit is prone to be increased.

In addition, the heating and humidifying mechanism inside the breathing circuit makes contact with the wall of the breathing circuit and the inspiratory gas flows over there, and thus there is a possibility of causing variation in the heating and humidification. Further, as shown in FIG. 6, there is also a possibility of causing a problem of developing condensation of the water vapor on the internal wall of the breathing circuit 102 and retaining the dew condensed water in the circuit 102.

In contrast, in the breathing circuit 20 according to the present invention shown in FIG. 2, the heating and humidification of the inspiratory gas is carried out using the entire circumference of the internal surface of the outer shell 4 of the artificial airway 2, so that heating and humidification sufficient for a user can be realized. In addition, since there is no substance impeding the flow of the inspiratory gas in the aeration region 12, there is no possibility of a ventilation control and airway pressure measurement going wrong. In addition, by suppressing the load on the inspiratory gas supply source 22, the running costs for the breathing circuit can be suppressed.

As described above, the artificial airway 2 according to the present invention and the breathing circuit 20 provided with the artificial airway 2 exhibit significant actions and effects as below.

The inspiratory gas can be heated and humidified in the artificial airway 2 arranged at a position near to a user, so that it is less affected by a change in temperature from the outside and risks of recondensing the water vapor in the artificial airway 2 can be reduced. In addition, it does not require excessive devices and members, such as a container for heating and humidification, a heater device to warm water in the heating and humidifying container, and a device for controlling the amount of water and the temperature, and excessive disposable tubes are also not required, so that the facility costs and the running costs can be reduced and risks of a tube connection failure and disengagement of a tube can also be reduced.

Further, heating and humidification of the inspiratory gas can be carried out using a large heating and humidifying area, such as the entire circumference of the internal surface of the outer shell 4 of the artificial airway 2, so that heating and humidification of the inspiratory gas sufficient for a user can be realized and condensation on the wall of the circuit does not develop as well. In addition, there is no excessive member for humidification in the artificial airway 2, so that there is no possibility of increasing the flow resistance of the inspiratory gas and also no possibility of the ventilation control and the airway pressure measurement going wrong.

Therefore, heating and humidification of the inspiratory gas sufficient for a user can be achieved with a simple configuration without increasing the flow resistance of the inspiratory gas in the artificial airway, and further being less affected by a change in temperature from the outside, and without developing condensation on the wall of the circuit.

(Range of Application of Artificial Airway According to the Invention and Breathing Circuit Provided with the Artificial Airway)

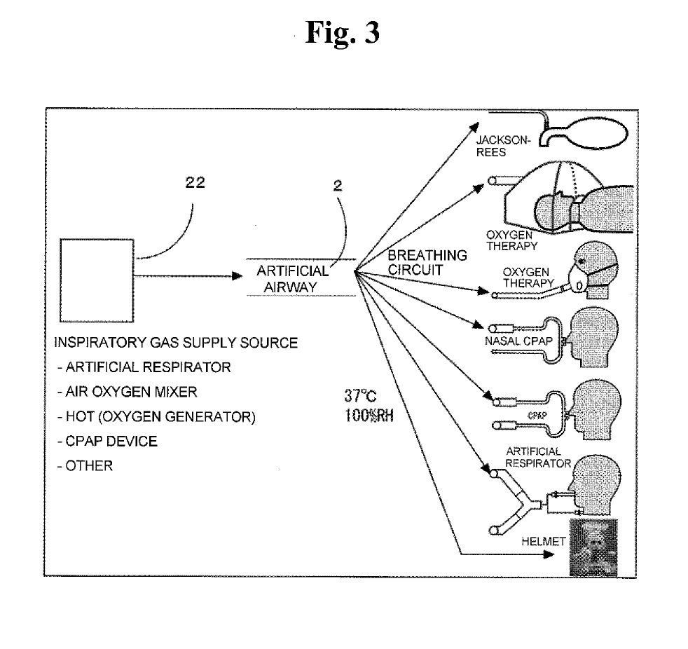

The artificial airway according to the present invention and the breathing circuit provided with the artificial airway are applicable to various fields, for example, as shown in FIG. 3 not limited to the applications in medical fields. In addition, also for the inspiratory gas supply source, various devices can be used as shown in FIG. 3 in accordance with the field of application.

(Description of Another Embodiment of Artificial Airway According to the Invention According to the Invention and Breathing Circuit Provided with the Artificial Airway)<

### Description of Another Embodiment (1) of Artificial Airway According to the Invention>

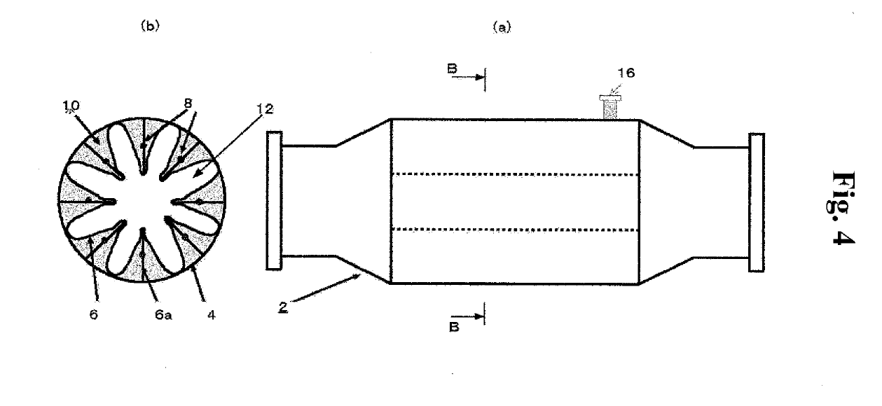

As another embodiment (1) of an artificial airway according to the present invention, a description is given to an embodiment of applying an artificial airway according to the present invention to an artificial nose using FIGS. 4(a) and 4(b). FIGS. 4(a) and 4(b) are schematic views illustrating a structure of an embodiment of an artificial airway (artificial nose) 2 according to the present invention, and FIG. 4(a) is a full view of the artificial airway (artificial nose) 2 taken from the side, and FIG. 4(b) is a cross-sectional view taken from the arrows B-B in FIG. 4(a).

Generally, an artificial nose is used at an end closest to a user of a breathing circuit and is one type of an artificial airway through which an inspiratory gas and an expiratory gas pass alternately in an aeration region. Normally, an artificial nose has one end in communication with an inspiratory tube (equivalent to the artificial airway 2 shown in FIGS. 1(a) and 1(b)) and with an expiratory tube via a Y shaped connector and has the other end used by being connected to an intratracheal tube of the user. This intratracheal tube is inserted to a patient from the nose (in a case of nasal intubation), the mouth (in a case of oral intubation), or the trachea (in a case of tracheal intubation). Thus, an inspiratory gas at a predetermined flow rate is supplied to the inspiratory tube by the inspiratory supply source, and the inspiratory gas passes through the inspiratory tube and the Y shaped connector and flows in the artificial nose 2 to be supplied to the user. The expiratory gas exhaled from the user flows in the artificial nose 2 and passes through the Y shaped connector and the expiratory tube to be discharged to the atmosphere.

Normally, the total length of the artificial airway (artificial nose) 2 is considerably shorter compared to that of the inspiratory tube (equivalent to the artificial airway 2 shown in FIGS. 1(a) and 1(b)), and there is a possibility of not allowing a sufficient area for the moisture permeable and water resistant film 6 to heat and humidify the inspiratory gas. For this reason, as described below in detail, in the present embodiment, the moisture permeable and water resistant film 6 has a wavy shape like the folds of a nasal cavity of a person in order to get a large heating and humidifying area in the shorter total length.

A basic configuration of the artificial airway (artificial nose) 2 of the present embodiment is provided with the outer shell 4 in an approximately cylindrical shape, the moisture permeable and water resistant film 6 in a folded shape disposed on the entire circumference the an internal surface of the outer shell 4, and the linear heater 8. Then, the water retention region 10 is formed between the outer shell 4 and the moisture permeable and water resistant film 6, and the aeration region 12 is formed on the internal surface side of the moisture permeable and water resistant film 6. The outer shell 4 is also provided with the feed water inlet 16 to supply water to the water retention region 10.

In a case that the moisture permeable and water resistant film 6 of the present embodiment had a shape same as that of the moisture permeable and water resistant film 6 of the artificial airway 2 shown in FIG. 1(b), the area of the moisture permeable and water resistant film making contact with the inspiratory gas would become smaller by the difference of the total lengths, so that there would be a possibility of not being able to heat and humidify sufficiently. With that, in the present embodiment, the moisture permeable and water resistant film 6 is made into folds inside the artificial nose 2 and thus a contact area of the moisture permeable and water resistant film 6 with the inspiratory gas is enlarged to heat and humidify sufficiently.

In addition, the conventional artificial nose has a heat and moisture exchanger element loaded in the aeration region, so that there are risks of an obstruction of the heat and moisture exchanger element due to sputum, blood, and the like of the patient, and also risks of rising the circuit resistance of the artificial nose by a water droplet clinging on the heat and moisture exchanger element. However, in the present embodiment, there is no heat and moisture exchanger element in the aeration region 12, so that there is no possibility of causing such a problem.

In the present embodiment, to form the moisture permeable and water resistant film 6 in a wavy shape, moisture permeable and water resistant film supporting struts 6a are attached on the internal surface of the outer shell 4, extending from the internal surface to a direction of the center of the circle. In the present embodiment, the linear heater 8 is provided in the water retention region 10, and specifically, the linear heater 8 is attached to the moisture permeable and water resistant film supporting struts 6a. It should be noted that it is not limited thereto, and it is also possible to, for example, dispose a linear heater outside the outer shell 4 and also to load a plate heater outside the outer shell 4.

As described above, in the present embodiment, the moisture permeable and water resistant film 6 has a wavy shape like the folds of a nasal cavity of a person, so that the area to heat and humidify inside the aeration region 12 can be increased drastically. This enables to achieve heating and humidification of the inspiratory gas sufficient for a user even with an artificial nose having a short total length.

### <Description of Another Embodiment (2) of Artificial Airway According to the Invention>

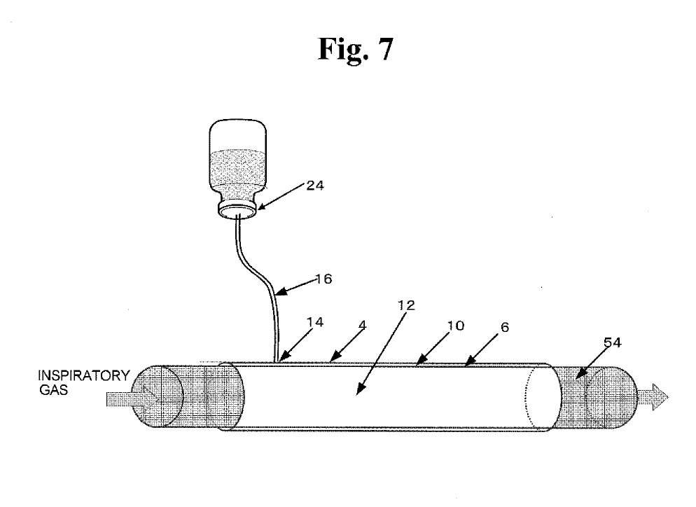

As another embodiment (2) of an artificial airway according to the present invention, a description is given to an artificial airway having a tubular reinforcement member disposed on an internal surface side of a moisture permeable and water resistant film using FIG. 7.

In FIG. 7, the artificial airway 2 is provided with the tubular outer shell 4 having air tightness and water tightness and the moisture permeable and water resistant film 6 disposed on the entire circumference of the internal surface of the outer shell 4, and further, a column net tube 54 made of a resin, which is a tubular reinforcement member, is disposed on the internal surface side of the moisture permeable and water resistant film 6 so as to make contact with the internal surface of the moisture permeable and water resistant film 6. With such a structure, the water retention region 10 is formed between the internal surface of the outer shell 4 and the outer surface of the moisture permeable and water resistant film 6, and the aeration region 12 is formed on the internal surface side of the moisture permeable and water resistant film 6 supported by the column net tube 54 made of a resin. Outside the outer shell 4, a heater 8 is disposed (not shown) and the water stored in the water container 24 is led into the water retention region 10 from the feed water inlet 14 through the water supply tube 16.

In the present embodiment, the resin column net tube is used as a tubular reinforcement member 54, using a resin material and being in a mesh shape, so that it is possible to realize a reinforcement member 54 of a light weight while having sufficient strength for actual use.

It should be noted that the tubular reinforcement member 54 is not limited to those made of a resin and can use any other material, including a metal, and the shape is also not limited to a cylindrical shape and can employ any other shape and also does not necessarily have a mesh.

According to the present embodiment, even in a case that the tube configured with the moisture permeable and water resistant film 6 does not have the strength for maintaining the cylindrical shape, the column net tube 54 made of a resin (tubular reinforcement member) is disposed so as to make contact with the internal surface of the moisture permeable and water resistant film 6, so that the tube configured with the moisture permeable and water resistant film 6 can be maintained in a cylindrical shape and the moisture permeable and water resistant film can be prevented from expanding inward to secure a sufficient size of the aeration region 12.

### <Description of Another Embodiment (3) of Artificial Airway According to the Invention>

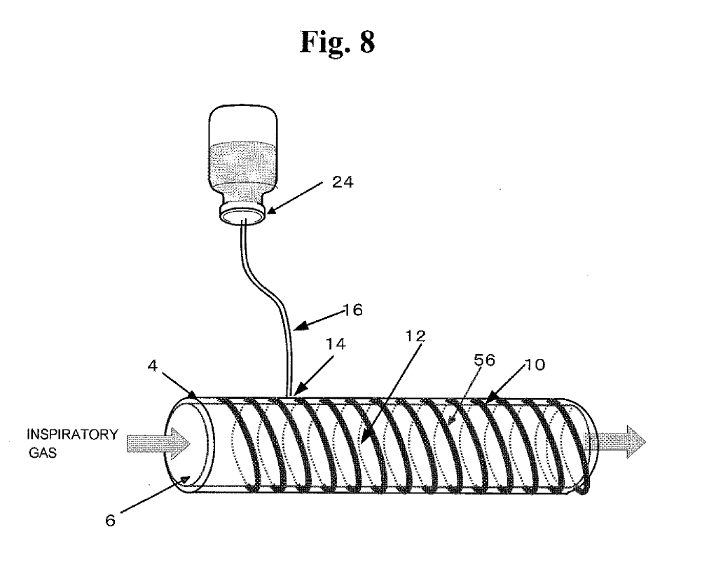

As another embodiment (3) of an artificial airway according to the present invention, a description is given to an artificial airway having a helical core disposed in the water retention region between an outer shell and a moisture permeable and water resistant film using FIG. 8.

In FIG. 8, the artificial airway 2 is provided with the tubular outer shell 4 having air tightness and water tightness and the moisture permeable and water resistant film 6 disposed on the entire circumference of the internal surface of the outer shell 4, and thus, the water retention region 10 is formed between the internal surface of the outer shell 4 and the outer surface of the moisture permeable and water resistant film 6, and the aeration region 12 is formed on the internal surface side of the moisture permeable and water resistant film 6. In the present embodiment, further, a helical core 56 made of a resin is disposed in the water retention region 10 between the outer shell 4 and the moisture permeable and water resistant film 6.

Outside the outer shell 4, a heater 8 is disposed (not shown), and water stored in the water container 24 is led into the water retention region 10 from the feed water inlet 14 through the water supply tube 16. At this time, a helical flow channel guided by the helical core 56 is formed in the water retention region 10, and the water supplied from the feed water inlet 14 can stream entirely in the water retention region 10 along this helical flow channel.

Although the helical core 56 of the present embodiment is made of a resin, it is not limited to that and any other material, including a metal, can be used, and the shape is also not limited to a cylindrical shape and any other shape can be employed.

To form this artificial airway 2, it can be realized by, for example, adhering the moisture permeable and water resistant film 6 to inside the helical core 56 and adhering the outer shell 4 to outside the helical core 56, and seal bonding the moisture permeable and water resistant film 6 and the outer shell 4 at both ends.

According to the present embodiment, even in a case that the tube configured with the moisture permeable and water resistant film 6 does not have the strength for maintaining the cylindrical shape, the helical core 56 is disposed in the water retention region 10, so that the tube configured with the moisture permeable and water resistant film 6 can be maintained in a cylindrical shape and the moisture permeable and water resistant film 6 can be prevented from expanding inward to secure a sufficient size of the aeration region 12. In addition, the water flows along the helical flow channel formed with the helical core 56, so that the helical core 56 does not impede the flow of water in the water retention region 10.

Embodiments of an artificial airway according to the present invention and a breathing circuit provided with the artificial airway are not limited to the above embodiments, and the present invention includes any other embodiments.

1. An artificial airway used for a breathing circuit, comprising:
a tubular outer shell;
a moisture permeable and water resistant film disposed on an entire circumference of an internal surface of the outer shell, forming a water retention region with the outer shell, and forming an aeration region on an internal surface side thereof;
a feed water inlet provided in the outer shell to supply water to the water retention region; and
a heater disposed outside the outer shell, heating the water in the water retention region to generate water vapor, and also heating an inspiratory gas flowing in the aeration region,
wherein the water supplied from the feed water inlet is retained in the water retention region by the moisture permeable and water resistant film, and only the water vapor generated by the heating of the heater passes through the moisture permeable and water resistant film and flows into the aeration region to heat and humidify the inspiratory gas flowing in the aeration region.

2. The artificial airway used for a breathing circuit according to claim 1, wherein the heater is disposed outside the outer shell in a region where the water retention region is formed.

3. The artificial airway used for a breathing circuit according to claim 1, wherein the heating and humidification of the inspiratory gas is possible to be adjusted at the same time by adjusting a power application to the heater.

4. The artificial airway used for a breathing circuit according to claim 1, wherein the moisture permeable and water resistant film is made of a resinous sheet or a resinous film.

5. The artificial airway used for a breathing circuit according to claim 1, wherein the moisture permeable and water resistant film includes a nonwoven fabric having moisture permeability and water resistance.

6. The artificial airway used for a breathing circuit according to claim 1, wherein the moisture permeable and water resistant film includes a porous material or a nonporous material.

7. The artificial airway used for a breathing circuit according to claim 1, wherein a tubular reinforcement member is disposed on the internal surface side of the moisture permeable and water resistant film to make contact with the internal surface.

8. The artificial airway used for a breathing circuit according to claim 1, wherein a helical core is disposed in the water retention region between the outer shell and the moisture permeable and water resistant film, and the water supplied from the feed water inlet flows along a helical flow channel formed with the helical core.

9. An artificial airway used for a breathing circuit, comprising:
an outer shell in an approximately cylindrical shape;
a moisture permeable and water resistant film, formed into folds, disposed on an entire circumference of an internal surface of the outer shell, forming a water retention region with the outer shell, and forming an aeration region on an internal surface side thereof;
a feed water inlet provided in the outer shell to supply water to the water retention region; and
a heater provided in the water retention region or outside the outer shell, heating the water in the water retention region to generate water vapor, and also heating an inspiratory gas flowing in the aeration region,
said artificial airway applicable as an artificial nose in which the inspiratory gas and an expiratory gas flow in the aeration region, wherein
the water supplied from the feed water inlet is retained in the water retention region by the moisture permeable and water resistant film, and only the water vapor generated by the heating of the heater passes through the moisture permeable and water resistant film and flows into the aeration region to heat and humidify the inspiratory gas flowing in the aeration region.

10. A breathing circuit, comprising:
the artificial airway according to claim 1;
an inspiratory gas supply source supplying the inspiratory gas to the aeration region of the artificial airway connected thereto; and
water supply means supplying the water to the water retention region with a basically constant static pressure via the feed water inlet, wherein
the water retention region is supplemented with water by the water supply means in an amount of water corresponding to an amount of water vapor passed through the moisture permeable and water resistant film and flown out.

11. The breathing circuit according to claim 10, wherein the water supply means supplies the water by dropping from a container that contains the water and includes:
drop rate measurement means measuring a rate of the dropping; and
control means carrying out a control process of issuing an alert, based on drop rate measurement data sent from the drop rate measurement means, when the drop rate exceeds a predetermined value or when the drop rate falls below a predetermined value.

12. The breathing circuit according to claim 11, further comprising temperature measurement means measuring a temperature of the inspiratory gas flowing in the aeration region in proximity of an exit of the inspiratory gas of the artificial airway, wherein the control means carrying out a control process of adjusting the power application of the heater based on temperature measurement data sent from the temperature measurement means.
